(19) United States
(12) Patent Application Publication
(10) Pub. No.: US 2011/0077482 A1
(43) Pub. Date: Mar. 31, 2011

(54) PORTABLE ELECTRONIC DEVICE AND METHOD FOR USING THE SAME

(75) Inventors: Hsing-Yuan Hsieh, Shindian (TW); Miao Zhao, Shenzhen (CN)

(73) Assignees: SHENZHEN FUTAIHONG PRECISION INDUSTRY CO., LTD., ShenZhen City (CN); FIH (HONG KONG) LIMITED, Kowloon (HK)

(21) Appl. No.: 12/783,036

(22) Filed: May 19, 2010

(30) Foreign Application Priority Data
Sep. 29, 2009 (CN) 200910307966.9

(51) Int. Cl. A61B 5/1455

(52) U.S. Cl. 600/323

(57) ABSTRACT

A portable electronic device having at least one function module selected from a group consisting of a wireless communication module, an image capturing module, and an audio/video file player module also includes a blood oxygen saturation (SaO₂) detector and a processor connected to the SaO₂ detector. The SaO₂ detector including a first light emitting diode (LED) emitting visible light, a second LED emitting infrared light, and a photoelectric sensor. The visible light of the first LED and the infrared light of the second LED irradiates a person's body, the photoelectric sensor receives visible light and infrared light reflected by the person's body and generates electrical signals corresponding to the reflected visible light and infrared light, and the processor calculates the SaO₂ of the person's body according to the electrical signals.

### BACKGROUND

1. Technical Field

The present disclosure relates to portable electronic devices, and particularly to a multifunctional portable electronic device having a built-in blood oxygen saturation (SaO₂) measuring device and a method for using the same.

2. Description of Related Art

SaO₂ is commonly expressed as a percentage of oxyhemoglobin (HbO₂) in the hemoglobin (Hb) in a person's body. During medical care, SaO₂ is an important physiological parameter and often needs to be measured. Generally, conventional SaO₂ measuring devices only have the single function of measuring SaO₂. If an SaO₂ measuring device is integrated with a more frequently used instrument, it may more convenient and cost effective.

Therefore, there is room for improvement within the art.

### BRIEF DESCRIPTION OF THE DRAWINGS

Many aspects of the present portable electronic device and method for using the same can be better understood with reference to the following drawings. The components in the various drawings are not necessarily drawn to scale, the emphasis instead being placed upon clearly illustrating the principles of the present portable electronic device and method for using the same. Moreover, in the drawings, like reference numerals designate corresponding parts throughout the figures.

FIG. 1 is a schematic view of a portable electronic device, according to an exemplary embodiment.

FIG. 2 is a block diagram of the portable electronic device shown in FIG. 1.

FIG. 3 is a flow chart of a method for using the portable electronic device shown in FIG. 1, according to an exemplary embodiment.

### DETAILED DESCRIPTION

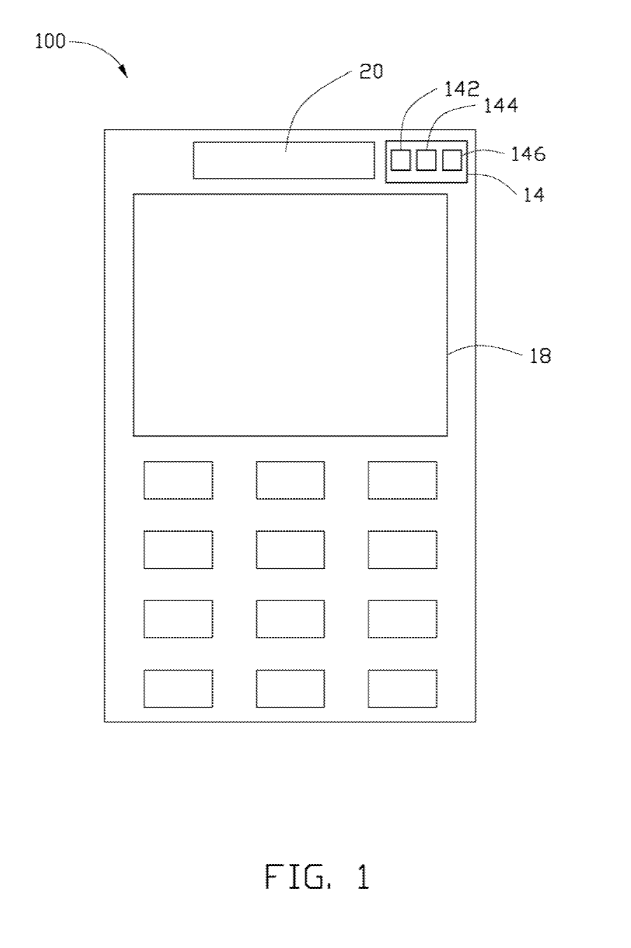

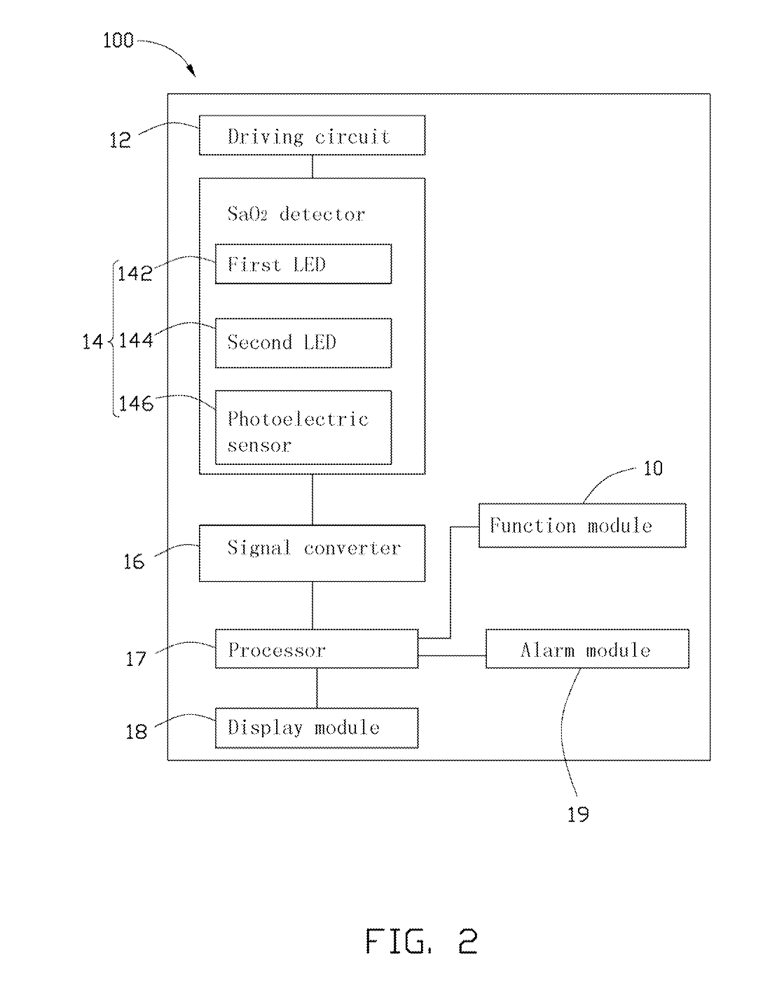

FIG. 1 schematically shows a portable electronic device 100, according to an exemplary embodiment. The device 100 can be a mobile phone, a digital camera, or a personal digital assistant (PDA), for example. Also referring to FIG. 2, the device 100 includes at least one conventional function module 10, such as a wireless communication module, an image capturing module, an audio/video file player module, etc., and further includes a driving circuit 12, an SaO₂ detector 14, a signal converter 16, a processor 17, a display module 18, and an alarm module 19. The driving circuit 12, the SaO₂ detector 14, the signal converter 16, and the processor 17 are electrically connected in series. The display module 18 and the alarm module 19 are both electrically connected to the processor 17.

At least a portion of the SaO₂ detector 14 is installed on an exterior surface of the portable electronic device 100. The SaO₂ detector 14 includes a first light emitting diode (LED) 142, a second LED 144, and a photoelectric sensor 146. The first LED 142 is configured to emit visible light, and the second LED 144 is configured to emit infrared light. The driving circuit 12 can drive the first LED 142 and the second LED 144 to emit light. The first LED 142 and the second LED 144 are positioned adjacent to the photoelectric sensor 146. When the SaO₂ detector 14 is positioned adjacent to an exposed portion of a person's body (i.e., skin), the driving circuit 12 drives the first LED 142 and the second LED 144 to respectively emit visible light and infrared light. When the visible light and the infrared light irradiate the exposed portion, some visible light and infrared light is absorbed by the exposed portion, and substantially the remaining visible light and infrared light are reflected back to the photoelectric sensor 146. The photoelectric sensor 146 can receive the reflected visible light and infrared light and generate electrical signals corresponding to the received light.

The signal converter 16 can receive the electrical signals generated by the photoelectric sensor 146, convert the electrical signals to digital signals, and transmit the digital signals to the processor 17. The processor 17 can be a conventional central processing unit (CPU) of the portable electronic device 100. The processor 17 can calculate the quantity of the reflected visible light and infrared light according to their corresponding electrical signals, and then calculate the quantity of the absorbed visible light and infrared light according to the quantity of the reflected visible light and infrared light. Since HbO₂ and Hb have different absorption rates of visible light and infrared light, the processor 17 can calculate the ratio of the quantity of HbO₂ with respect to the quantity of Hb according to the quantity of the absorbed visible light and infrared light and known absorption rates of visible light and infrared light of HbO₂ and Hb, thereby obtaining the SaO₂ of the person. The processor 17 can also be connected to the function module 10 to control the function module 10 to work.

The display module 18 can be a conventional display of the portable electronic device 100. The alarm module 19 can be a conventional indicator light, bell, or vibrator of the portable electronic device 100. The processor 17 can control the display module 18 and the alarm module 19 to function. When obtaining the SaO₂ of the person, the processor 17 can transmit the SaO₂ data to the display module 18 for display. A predetermined range of SaO₂ can be set and stored in the processor 17. If the SaO₂ is out of the predetermined range, the processor 17 can control the alarm module 19 to give the alarm by means of shining, ringing, or vibrating.

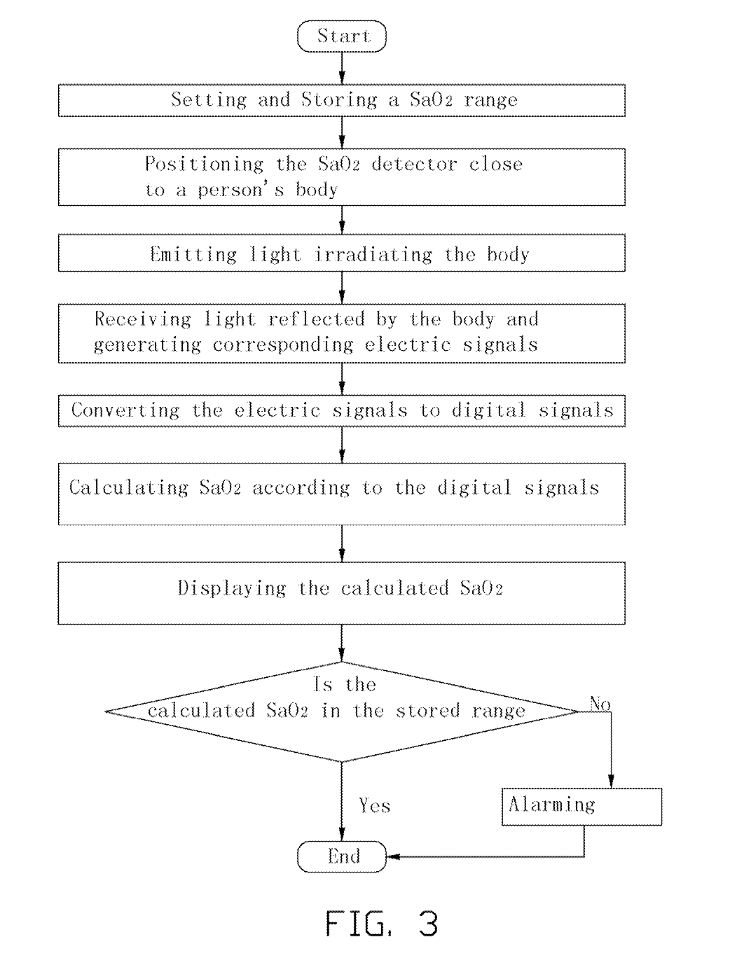

Also referring to FIG. 3, a method for using the portable electronic device 100 to measure a person's SaO₂, according to an exemplary embodiment, is provided. The method may at least include the following steps.

First, a predetermined range of SaO₂ is set and stored in the processor 17. The portable electronic device 100 is positioned adjacent to an exposed portion of a person's body, such as a finger or a forearm, and the SaO₂ detector 14 is positioned as close to the exposed portion of the body as possible to maintain the accuracy of the detector 14 by preventing the dissipation of reflected light. Thus, the driving circuit 12 is actuated and drives the first LED 142 and the second LED 144 to emit light. When the visible light and the infrared light irradiate the exposed portion of the person's body, some visible light and infrared light is absorbed by the exposed body portion, and substantially the remaining visible light and infrared light are reflected back to the photoelectric sensor 146.

The photoelectric sensor 146 receives the reflected visible light and infrared light and generates electrical signals corresponding to the received light. The signal converter 16 receives the electrical signals generated by the photoelectric sensor 146, converts the electrical signals to digital signals, and transmits the digital signals to the processor 17.

When receiving the digital signals corresponding to the reflected visible light and infrared light, the processor 17 calculates the quantity of the reflected visible light and infrared light according to their corresponding electrical signals, and then calculates the quantity of the absorbed visible light and infrared light according to the quantity of the reflected visible light and infrared light. Thus, the processor 17 further calculates the ratio of the quantity of HbO₂ with respect to the quantity of Hb according to the quantity of the absorbed visible light and infrared light and known absorption rates of visible light and infrared light of HbO₂ and Hb, thereby obtaining the SaO₂ of the person.

When obtaining the SaO₂ of the person, the processor 17 transmits the SaO₂ data to the display module 18 to display. If the SaO₂ is out of the predetermined range, the processor 17 controls the alarm module 19 to give the alarm, thereby reminding the examiner and the examined person.

The present portable electronic device 100 integrated with the SaO₂ measuring device (i.e., the subassembly including at least the driving circuit 12, the SaO₂ detector 14, the signal converter 16, the processor 17, the display module 18, and an alarm module 19) is easy to be carried and used. The users of the portable electronic device 100 need not carry a separate SaO₂ measuring device, which can decrease the burden of the users and conserve cost.

It is to be further understood that even though numerous characteristics and advantages of the present embodiments have been set forth in the foregoing description, together with details of structures and functions of various embodiments, the disclosure is illustrative only, and changes may be made in detail, especially in matters of shape, size, and arrangement of parts within the principles of the present invention to the full extent indicated by the broad general meaning of the terms in which the appended claims are expressed.

What is claimed is:

1. A portable electronic device, comprising:
at least one function module selected from a group consisting of a wireless communication module, an image capturing module, and an audio/video file player module;
a blood oxygen saturation (SaO₂) detector, the SaO₂ detector including a first light emitting diode (LED) emitting visible light, a second LED emitting infrared light, and a photoelectric sensor; and
a processor connected to the function module and the SaO₂ detector; wherein the processor controls the function module to work, and the visible light of the first LED and the infrared light of the second LED irradiate a person's body, the photoelectric sensor receiving visible light and infrared light reflected by the person's body and generating electrical signals corresponding to the reflected visible light and infrared light, the processor calculating the SaO₂ of the person's body according to the electrical signals.

2. The portable electronic device as claimed in claim 1, further comprising a driving circuit connected to the SaO₂ detector to drive the first LED and the second LED to emit light.

3. The portable electronic device as claimed in claim 1, further comprising a signal converter, the SaO₂ detector connected to the processor through the signal converter, the signal converter converting the electrical signals generated by the photoelectric sensor to digital signals and transmitting the digital signals to the processor, and the processor calculating the SaO₂ of the person's body according to the digital signals.

4. The portable electronic device as claimed in claim 1, wherein the processor calculates the quantity of the reflected visible light and infrared light according to their corresponding electrical signals, calculates the quantity of the absorbed visible light and infrared light according to the quantity of the reflected visible light and infrared light, and further calculates the ratio of the quantity of oxyhemoglobin (HbO₂) with respect to the quantity of hemoglobin (Hb) according to the quantity of the visible light and infrared light absorbed and known absorption rates of visible light and infrared light by HbO₂ and Hb, thereby obtaining the SaO₂ of the person's body.

5. The portable electronic device as claimed in claim 1, further comprising a display module connected to the processor to display the SaO₂ of the person's body calculated by the processor.

6. The portable electronic device as claimed in claim 1, further comprising an alarm module connected to the processor to alarm when the SaO₂ of the person's body calculated by the processor is out of a predetermined range.

7. A method for using a portable electronic device having at least one function module selected from a group consisting of a wireless communication module, an image capturing module, and an audio/video file player module to measure the blood oxygen saturation (SaO₂) of a person's body, comprising:
using the portable electronic device to irradiate a person's body with visible light and infrared light emissions;
receiving the visible light and infrared light reflected by the person's body and generating electrical signals corresponding to the received visible light and infrared light; and
calculating the SaO₂ of the person's body according to the electrical signals.

8. The method as claimed in claim 7, wherein the step of calculating the SaO₂ of the person's body according to the electrical signals includes these substeps:
calculating the quantity of the received visible light and infrared light according to their corresponding electrical signals;
calculating the quantity of the emitted visible light and infrared light irradiated on the person's body that is absorbed by the person's body according to the quantity of the received visible light and infrared light; and
calculating the ratio of the quantity of oxyhemoglobin (HbO₂) with respect to the quantity of hemoglobin (Hb) according to the quantity of the absorbed visible light and infrared light and known absorption rates of visible light and infrared light by HbO₂ and Hb, thereby obtaining the SaO₂ of the person's body.

9. The method as claimed in claim 7, further comprising activating an alarm when the SaO₂ of the person's body is out of a predetermined range.
